(19) United States
(12) Patent Application Publication
(10) Pub. No.: US 2024/0359014 A1
(43) Pub. Date: Oct. 31, 2024

(54) SYSTEMS AND METHODS FOR SENSED SIGNAL GUIDANCE

(71) Applicant: Boston Scientific Neuromodulation Corporation, Valencia, CA (US)

(72) Inventors: G. Karl Steinke, Valencia, CA (US); Soroush Niketeghad, Stevenson Ranch, CA (US); Mahsa Malekmohammadi, Sherman Oaks, CA (US)

(21) Appl. No.: 18/646,260

(22) Filed: Apr. 25, 2024

Related U.S. Application Data
(60) Provisional application No. 63/461,771, filed on Apr. 25, 2023.

(51) Int. Cl. A61N 1/36; A61N 1/05; A61N 1/372; G16H 20/40

(52) U.S. Cl. CPC A61N 1/36135; A61N 1/0534; A61N 1/36082; A61N 1/36132; A61N 1/37247; A61N 1/37252; G16H 20/40

(57) ABSTRACT

A system may include an electrostimulator configured to provide electrostimulation to a neural target patient via electrodes on a lead, a sensing circuit configured to sense, at a plurality of sensing locations, evoked responses (ERs) to the electrostimulation, a user interface, and a controller operably connected to the electrostimulator, the sensing circuit and the user interface. The controller may be configured to deliver the electrostimulation in accordance with a stimulation setting, determine the sensed ERs to the electrostimulation delivered in accordance with a sensing setting and the stimulation setting, determine acceptance criteria using user input received via the user interface, compare the sensed ERs to the acceptance criteria to provide a comparison result, and display on the user interface an indicator of the comparison result.

### CLAIM OF PRIORITY

This application claims the benefit of U.S. Provisional Application No. 63/461,771 filed on Apr. 25, 2023, which is hereby incorporated by reference in its entirety.

### TECHNICAL FIELD

This document relates generally to medical systems, and more particularly, but not by way of limitation, to systems, devices, and methods for using sensed evoked responses to guide neurostimulation.

### BACKGROUND

Medical devices may include therapy-delivery devices configured to deliver a therapy to a patient and/or monitors configured to monitor a patient condition via user input and/or sensor(s). For example, therapy-delivery devices for ambulatory patients may include wearable devices and implantable devices, and further may include, but are not limited to, stimulators (such as electrical, thermal, or mechanical stimulators) and drug delivery devices (such as an insulin pump). An example of a wearable device includes, but is not limited to, transcutaneous electrical neural stimulators (TENS), such as may be attached to glasses, an article of clothing, or a patch configured to be adhered to skin. Implantable stimulation devices may deliver electrical stimuli to treat various biological disorders, such as pacemakers to treat cardiac arrhythmia, defibrillators to treat cardiac fibrillation, heart failure cardiac resynchronization therapy devices, cochlear stimulators to treat deafness, retinal stimulators to treat blindness, muscle stimulators to produce coordinated limb movement, spinal cord stimulators (SCS) to treat chronic pain, cortical and Deep Brain Stimulators (DBS) to treat motor and psychological disorders, Peripheral Nerve Stimulation (PNS), Functional Electrical Stimulation (FES), and other neural stimulators to treat urinary incontinence, sleep apnea, shoulder subluxation, etc. A neurostimulation device (e.g., DBS, SCS, PNS or TENS) may be configured to treat pain. By way of example and not limitation, a DBS system may be configured to treat tremor, bradykinesia, and dyskinesia and other motor disorders associated with Parkinson's Disease (PD).

It has been proposed to use evoked potentials to guide lead placement and/or programming to match a patient's evoked potentials to a desired or “target” response. For example, Evoked Resonant Neural Activity (ERNA) has been proposed as a feedback signal for STN DBS therapy for Parkinson's disease. (See Thevathasan W, Sinclair NC, Bulluss KJ and McDermott HJ (2020) Tailoring Subthalamic Nucleus Deep Brain Stimulation for Parkinson's Disease Using Evoked Resonant Neural Activity. Front. Hum. Neurosci. 14:71. doi: 10.3389/fnhum.2020.00071.) ERNA may also be referred to by other names such as DBS Local Evoked Potentials (DLEP). Evoked potentials, including ERNA or DLEPs, may be present in other indications and anatomical structures or locations.

It is desired to improve the lead placement and/or programming using ERNA or other evoked responses.

### SUMMARY

The present inventors have recognized that the desired evoked response target varies. For example, the ERNA target for STN DBS may vary among patients. The present inventors recognize that the ERNA target, or other evoked response target, may vary based on anatomical target and trajectory of the lead placement and can vary with other factors such as the evoking and recording settings, the surgery center, the surgeon, the surgical or programming techniques, preferences, and errors. Embodiments of the present subject matter provide systems, device and methods that implement user-selected acceptance criteria to accommodate for different desired evoked response targets.

An example (e.g., “Example 1”) of a system may include an electrostimulator configured to provide electrostimulation to a neural target patient via electrodes on a lead, a sensing circuit configured to sense, at a plurality of sensing locations, evoked responses (ERs) to the electrostimulation, a user interface, and a controller operably connected to the electrostimulator, the sensing circuit and the user interface. The controller may be configured to deliver the electrostimulation in accordance with a stimulation setting, determine the sensed ERs to the electrostimulation delivered in accordance with a sensing setting and the stimulation setting, determine acceptance criteria using user input received via the user interface, compare the sensed ERs to the acceptance criteria to provide a comparison result, and display on the user interface an indicator of the comparison result.

In Example 2, the subject matter of Example 1 may optionally be configured such that the electrostimulator is configured to provide electrostimulation to a therapy target or other preferred lead placement location in a brain of the patient via a deep brain stimulator (DBS) lead. The DBS lead may be a directional lead and at least some of the electrodes on the directional lead may be radially segmented electrodes. The evoked responses may include Evoked Resonant Neural Activity (ERNA). In some embodiments, the lead may be a non-directional lead.

In Example 3, the subject matter of any one or more of Examples 1-2 may optionally be configured such that the controller is configured to receive, using the user interface, user-provided acceptance bounds for the sensed ERs, and the acceptance criteria include the user-provided acceptance bounds. For example, the user-provided acceptance bounds may include details and parameters such as may be used for scoring and weighting.

In Example 4, the subject matter of any one or more of Examples 1-2 may optionally be configured to further include a storage system configured to store a plurality of ER templates. The ER templates may each represent one or more of a single, patient-specific response or a population-based response to electrostimulation of the neural target. The user input received via the user interface may include a user selection of a target ER template from the stored ER templates. The acceptance criteria may include the target ER template. The controller may be configured to compare the sensed ERs to the target ER template to provide the comparison result The controller may, by way of example and not limitation, provide scores or recommendations based on the comparison.

In Example 5, the subject matter of Example 4 may optionally be configured such that the system includes an operating room (OR) system configured to be used during a lead placement procedure where the OR system includes at least the electrostimulator, the sensing circuit and the user interface. The OR system may include storage, and the storage system may include storage within the OR system. The storage system may include a cloud-based storage in a cloud-computing system. By way of example and not limitation, the subject matter may include exchanging information between computers via the cloud, uploading measurements to the cloud such as may be used to implement processes for template comparisons and pushing results down to computer(s) for display to user, retrieving templates from the cloud.

In Example 6, the subject matter of any one or more of Examples 1-5 may optionally be configured such that the controller is configured to display on the user interface representations of the acceptance criteria and the sensed ERs and/or derivatives thereof.

In Example 7, the subject matter of any one or more of Examples 1-6 may optionally be configured such that the controller is configured to display on the user interface a representation of the lead, and optionally determine and display a suggested lead movement to cause the sensed ERs to compare more favorably to the acceptance criteria and in so doing conform better to a user desired lead location or outcome.

In Example 8, the subject matter of any one or more of Examples 1-7 may optionally be configured such that the controller is configured to determine and display on the user interface a suggestion for initializing and/or changing the stimulation setting or the sensing setting to cause the sensed ERs to compare more favorably to the acceptance criteria.

In Example 9, the subject matter of any one or more of Examples 1-8 may optionally be configured to further include an ER template creator configured to provide ER templates available for user selection by modifying ER templates or creating new ER templates based on sensed ERs. The acceptance criteria may include a user-selected ER template, and the ER template creator may be configured to implement a machine learning algorithm to modify or create the ER templates by determining relationships among data where the data include the sensed ERs, and at least one lead implant procedure input, and determine the sensed ERs that corresponds to a desirable patient outcome when electrostimulation is delivered to the neural target. Optionally, the modification or creation of the ER templates may further be based on patient outcome for one or more patients, where the machine learning algorithm may determine relationships among data that further includes the patient outcomes. It is noted that some embodiments of the subject matter may create templates without the use of machine learning or artificial intelligence.

In Example 10, the subject matter of Example 9 may optionally be configured such that the ER template creator includes a cloud-based application to receive the data and implement the machine learning algorithm to modify or create the ER templates for the least one lead implant procedure input, or the OR system includes the ER template creator.

In Example 11, the subject matter of any one or more of Examples 1-10 may optionally be configured such that the acceptance criteria are indexed by region, clinical institution, group, or participants information. The target ER template may be selected from the ER data based at least in part on an identification of an institution where the patient is implanted and/or is treated with the electrostimulation, a group, or the participants information.

In Example 12, the subject matter of any one or more of Examples 1-11 may optionally be configured such that the stored ER templates are indexed by implanter information. The ER templates may each generated using ER data of patients treated by the same implanter. The target ER template may be selected from the stored ER templates based at least in part on an identification of an implanter that implants the lead.

In Example 13, the subject matter of any one or more of Examples 1-12 may optionally be configured such that the stored ER templates are indexed by symptom relief goals. The ER templates may each generated using ER data of patients experiencing improvement in the same symptom. The target ER template may be selected from the stored ER templates based at least in part on the sensed indication of symptom relief of the patient. The symptom relief goals may include at least one of a type motor function or a type of cognitive function. Symptom relief may optionally be paired with lead anatomical target or other factors.

In Example 14, the subject matter of any one or more of Examples 1-13 may optionally be configured such that the target ER template includes a target distribution of ER data and the sensing circuit is configured to determine a distribution of sensed ERs. The controller may be configured to compare the distribution of sensed ERs to the distribution of ER data. The user interface may be configured to display the distribution of sensed ERs and the target distribution of ER data.

In Example 15, the subject matter of any one or more of Examples 1-14 may optionally be configured such that the target ER template includes at least one target representative ER feature, and the sensing circuit is configured to determine at least one measured ER feature from the sensed ERs. The controller may be configured to compare the at least one target representative ER feature to the at least one measured ER feature. Optionally, the subject matter may include filtering/grouping by the evoke/record settings used to make measurements.

Example 16 includes subject matter (such as a method, means for performing acts, machine readable medium including instructions that when performed by a machine cause the machine to perform acts, or an apparatus to perform). The subject matter may include delivering, using a controller, electrostimulation from an electrostimulator in accordance with a stimulation setting, determining, using the controller and a sensing circuit configured to sense evoked responses (ERs), sensed ERs to the electrostimulation delivered in accordance with a sensing setting and the stimulation setting, determining acceptance criteria using user input received via a user interface, comparing, using the controller, the sensed ERs to the acceptance criteria to provide a comparison result, and displaying an indicator of the comparison result on the user interface.

In Example 17, the subject matter of Example 16 may optionally be configured such that the electrostimulator is configured to provide electrostimulation to a therapy target in a brain of the patient via a deep brain stimulator (DBS) lead. The evoked responses include Evoked Resonant Neural Activity (ERNA). The DBS lead may be a directional lead and at least some of the electrodes on the directional lead may be radially segmented electrodes. In some embodiments, the lead may be a non-directional lead.

In Example 18, the subject matter of any one or more of Examples 16-17 may optionally be configured to further include receiving, using the user interface, user-provided acceptance bounds for the sensed ERs, wherein the acceptance criteria include the user-provided acceptance bounds. For example, the user-provided acceptance bounds may include details and parameters such as may be used for scoring and weighting.

In Example 19, the subject matter of any one or more of Examples 16-18 may optionally be configured to further include receiving a user selection of a target ER template from stored ER templates. The acceptance criteria may include the target ER template, and the comparing may include comparing the sensed ERs to the target ER template to provide the comparison result. The controller may, by way of example and not limitation, provide scores or recommendations based on the comparison.

In Example 20, the subject matter of any one or more of Examples 16-19 may optionally be configured such that the stored ER templates are stored within storage of an operating room (OR) system configured to be used during a lead placement procedure, the OR system including at least the electrostimulator, the sensing circuit, the user interface, or the stored ER templates are stored in a cloud-based storage in a cloud-computing system. By way of example and not limitation, the subject matter may include exchanging information between computers via the cloud, uploading measurements to the cloud such as may be used to implement processes for template comparisons and pushing results down to computer(s) for display to user, retrieving templates from the cloud.

In Example 21, the subject matter of any one or more of Examples 16-20 may optionally be configured such that the displaying the indicator of the comparison result includes displaying on the user interface representations of the acceptance criteria and the sensed ERs and/or derivatives thereof.

In Example 22, the subject matter of any one or more of Examples 16-21 may optionally be configured to further include displaying on the user interface a representation of the lead, and determining and displaying a suggested lead movement to cause the sensed ERs to compare more favorably to the acceptance criteria.

In Example 23, the subject matter of any one or more of Examples 16-22 may optionally be configured to further include determining and displaying on the user interface a suggestion for initializing and/or changing the stimulation setting or the sensing setting to cause the sensed ERs to compare more favorably to the acceptance criteria.

In Example 24, the subject matter of any one or more of Examples 16-23 may optionally be configured to further include providing ER templates available for user selection by modifying ER templates or creating new ER templates based on sensed ERs and patient outcome for one or more patients. The acceptance criteria may include a user-selected ER template. Providing ER templates may include implementing one or more machine learning algorithms to modify or create the ER templates by determining relationships among data where the data include the sensed ERs, the patient outcomes, and at least one lead implant procedure input, and determine the sensed ERs that corresponds to a desirable patient outcome when electrostimulation is delivered to the neural target. The modification or creation of the ER templates based on patient outcome may be optional. It is noted that some embodiments of the subject matter may create templates without the use of machine learning or artificial intelligence.

In Example 25, the subject matter of Example 24 may optionally be configured such that the machine learning algorithm is implemented using at least one of a cloud-based application or a deployed system. The deployed system may be an operating room (OR) or in-clinic system.

In Example 26, the subject matter of any one or more of Examples 16-25 may optionally be configured to include indexing the acceptance criteria by region, clinical institution, group, or participants information. The acceptance criteria may be based at least in part on an identification of an institution where the patient is implanted or treated with the electrostimulation, a group, or the participants information.

In Example 27, the subject matter of any one or more of Examples 16-26 may optionally be configured to include indexing the acceptance criteria by implanter information. The acceptance criteria may be based at least in part on an identification of an implanter that implants the lead.

In Example 28, the subject matter of any one or more of Examples 16-27 may optionally be configured to include indexing the acceptance criteria by symptom relief goals. The acceptance criteria may be based at least in part on the sensed indication of symptom relief of the patient. The symptom relief goals may include at least one of a type motor function or a type of cognitive function. Symptom relief may optionally be paired with lead anatomical target or other factors.

In Example 29, the subject matter of any one or more of Examples 16-28 may optionally be configured such that the acceptance criteria include a target distribution of ER data. Determining sensed ERs may include determining a distribution of sensed ERs. Comparing may include comparing the distribution of sensed ERs to the distribution of ER data. Displaying the indicator may include displaying the distribution of sensed ERs and the target distribution of ER data.

In Example 30, the subject matter of any one or more of Examples 16-29 may optionally be configured such that the acquisition criteria include at least one target representative ER feature. Determining sensed ERs includes determining at least one measured ER feature from the sensed ERs. Comparing includes comparing the at least one target representative ER feature to the at least one measured ER feature. Optionally, the subject matter may include filtering/grouping by the evoke/record settings used to make measurements.

Example 31 includes a non-transitory machine-readable medium including instructions, which when executed by a machine, cause the machine to perform a method that includes delivering, using a controller, electrostimulation from an electrostimulator in accordance with a stimulation setting, determining, using the controller and a sensing circuit configured to sense evoked responses (ERs), sensed ERs to the electrostimulation delivered in accordance with a sensing setting and the stimulation setting, determining acceptance criteria using user input received via a user interface, comparing, using the controller, the sensed ERs to the acceptance criteria to provide a comparison result, and displaying an indicator of the comparison result on the user interface.

In Example 32, the subject matter of Example 31 may optionally be configured such that the method further includes receiving, using the user interface, user-provided acceptance bounds for the sensed ERs. The acceptance criteria may include the user-provided acceptance bounds. For example, the user-provided acceptance bounds may include details and parameters such as may be used for scoring and weighting.

In Example 33, the subject matter of any one or more of Examples 31-32 may optionally be configured such that the method further includes receiving a user selection of a target ER template from stored ER templates. The acceptance criteria may include the target ER template. Comparing may include comparing the sensed ERs to the target ER template to provide the comparison result. Scores or recommendations may be provided based on the comparison.

In Example 34, the subject matter of any one or more of Examples 31-33 may optionally be configured such that the acceptance criteria include a target distribution of ER data. The determining sensed ERs may include determining a distribution of sensed ERs. Comparing may include comparing the distribution of sensed ERs to the distribution of ER data. Displaying the indicator may include displaying the distribution of sensed ERs and the target distribution of ER data.

In Example 35, the subject matter of any one or more of Examples 31-34 may optionally be configured such that the acquisition criteria may include at least one target representative ER feature. Determining sensed ERs may include determining at least one measured ER feature from the sensed ERs. Comparing may include comparing the at least one target representative ER feature to the at least one measured ER feature. Optionally, the subject matter may include filtering/grouping by the evoke/record settings used to make measurements.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an electrical stimulation system, which may be used to deliver DBS.

FIG. 2 illustrates, by way of example and not limitation, an implantable pulse generator (IPG) in a DBS system.

FIGS. 3A-3B illustrate, by way of example and not limitation, leads that may be coupled to the IPG to deliver electrostimulation such as DBS.

FIG. 4 illustrates, by way of example and not limitation, a computing device for programming or controlling the operation of an electrical stimulation system.

FIG. 5 illustrates, by way of example, an example of an electrical therapy-delivery system.

FIG. 6 illustrates, by way of example and not limitation, a monitoring system and/or the electrical therapy-delivery system of FIG. 5, implemented using an IMD.

FIG. 7 illustrates a system for comparing sensed ERs to electrical stimulation against acceptance criteria.

FIG. 8 illustrates the system that may be used to implement the present subject matter.

FIG. 9 illustrates, by way of example and not limitation, some user interface functionality according to various embodiments of the present subject matter.

FIG. 10 illustrates, by way of example and not limitation, an operating room system.

FIG. 11 illustrates a system configured for use to compare ER data to a specific target response, and to update the target response based on a number of factors.

FIG. 12 illustrates by way of example and not limitation ERs when the external global pallidus (GPe), internal global pallidus (GPi) and subthalamic nucleus (STN) is stimulated.

FIG. 13 illustrates, by way of example and not limitation, a display for user interface that creates a response fit from a distribution of ERs.

FIGS. 14-1 and 14-2 illustrate, by way of example and not limitation, embodiments for measuring and matching ERs to acceptance criteria such as a template.

FIG. 15 illustrates, by way of example and not limitation, ER distributions for DBS lead electrodes.

FIGS. 16-1 and 16-2 illustrate, by way of example and not limitation, a library for ER targets.

FIG. 17 illustrates, by way of example and not limitation, an example of a view on a user interface for identifying a user-desired location for the sensed response target, the distribution of the sensed ERs, and a calculated maximum within the distribution of sensed ERs.

FIG. 18 illustrates, by way of example and not limitation, an ER target distribution and a measured or sensed ER distribution.

FIG. 19 illustrates, by way of example and not limitation, a method for using sensed ERs.

FIG. 20 illustrates, by way of example and not limitation, a method for determining an ER target and comparing sensed ERs to the determined ER target.

FIG. 21 illustrates, by way of example and not limitation, a display on a user interface for identifying an ER target, a sensed ER including a calculated maximum for the sensed ER, and a recommendation.

FIG. 22 illustrates, by way of example, elements that may be presented on a display in a user interface that provide a simple and clear confirmation of lead position.

FIG. 23 illustrates that the ER target may be specific to the surgery center used to perform the DBS lead implant.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

There is no single universal “optimal” stimulation target and desired response because the optimal target may vary depending upon condition or disorder, anatomy (anatomical target), lead and trajectory, center (imaging equipment position, etc.), surgeon (how they implant and position lead), or symptoms to improve (e.g., tremor to cognitive skill improvement). Raw responses may be displayed and the user may determine fitness based on the displayed raw responses. The user may adjust feature target bounds, acceptance thresholds and maximum number of steps before convergence.

ERs may be used to guide the implantation procedure or programming of parameter settings. The ERs may be caused by stimulation (e.g., pulses) that provides evoked potentials, stimulation that provides therapy and stimulation that can both deliver therapy and provide ERs. Stimulation may be located (1) where placing evoking pulses gets a desired response such as to maximize ERNA, (2) where listening for responses gets a desired response (e.g., maximize ERNA, (3) where placing lead is desired (e.g., best for therapy, and (4) where placing stimulation on the lead is desired (e.g., maximize therapy and/or minimize/counter side effects). Responses may be modulated by the details of the sensing, including amplifier settings, relationships between stimulating and sensing electrodes, natures of stimulating or sensing electrodes including geometry and surface among other factors, and signal processing occurring during and after measurement, including treatment within analogue or digital hardware, firmware, or software. The target may be an anatomical targe such as like a volume (e.g., STN), a collection of fibers, a sub-region (motor STN; dorsolateral STN), a volume of interest described within or related to a particular patient's brain such as from atlas or aggregate prior information, or a “point” that may be described by optimizing a stimulation location (e.g., one of (1)-(4) above).

Various embodiments may store fingerprints, templates, preferred, exemplary, or target responses (generally referred to as ER targets) for different centers, anatomies, or other causes for the different targets. The ER target may be specific, by way of example and not limitation, for anatomy, center/institutions, or surgeon. For example, there can be center-to-center differences because of how differences in how they place leads and collect data (imaging, angle, etc.) Each center may use its own center-specific ER target. Each target response may contain information concerning the distribution of data for ERs from a suite of measurements from a lead and involving a plurality of electrodes (each serially or in parallel evoking potentials and recording potentials). An ER target may be selected to be specific for a particular patient at a particular center. The system may use patient response or imaging data to determine what ER characteristics are important in determining the correct ER target to use.

Various embodiments are configured to learn from prior attempts to place lead and/or program the electrostimulator. The data analytics may be performed locally on the deployed device and/or performed remotely in centralized hubs (e.g., cloud computing). Delivered outputs based on the analytics may include insight reports, suggestions and/or guides. That is, various embodiments of the present subject matter afford many ER targets using a user interface, a library of targets, and/or local, remote, or distributed machine learning to learn the target.

This disclosure refers to an ERNA target for STN DBS, such as may be used to treat Parkinson's Disease, as a nonlimiting example of an ER to electrostimulation provided by an electrostimulator. The present subject matter may be applied for other ERs to other electrostimulation.

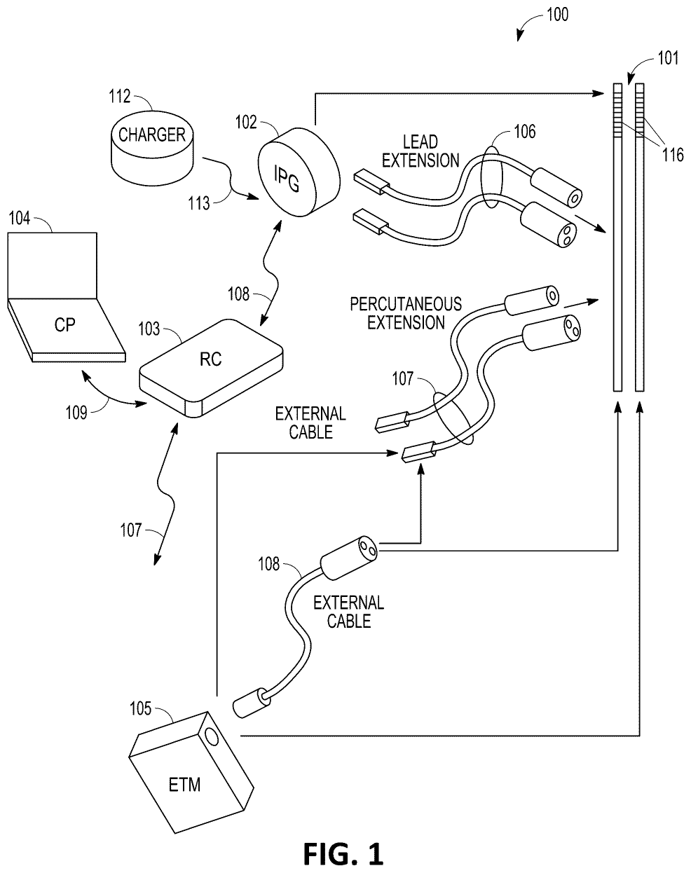

FIG. 1 illustrates, by way of example and not limitation, an electrical stimulation system 100, which may be used to deliver DBS. The electrical stimulation system 100 may generally include a one or more (illustrated as two) of implantable neuromodulation leads 101, a waveform generator such as an implantable pulse generator (IPG) 102, an external remote controller (RC) 103, a clinician programmer (CP) 104, and an external trial modulator (ETM) 105. The IPG 102 may be physically connected via one or more percutaneous lead extensions 106 to the neuromodulation lead(s) 101, which carry a plurality of electrodes 116. The electrodes, when implanted in a patient, form an electrode arrangement. As illustrated, the neuromodulation leads 101 may be percutaneous leads with the electrodes arranged in-line along the neuromodulation leads or about a circumference of the neuromodulation leads. Any suitable number of neuromodulation leads can be provided, including only one, as long as the number of electrodes is greater than two (including the IPG case function as a case electrode) to allow for lateral steering of the current. Alternatively, a surgical paddle lead can be used in place of one or more of the percutaneous leads. The IPG 102 includes pulse generation circuitry that delivers electrical modulation energy in the form of a pulsed electrical waveform (i.e., a temporal series of electrical pulses) to the electrodes in accordance with a set of modulation parameters.

The ETM 105 may also be physically connected via the percutaneous lead extensions 107 and external cable 108 to the neuromodulation lead(s) 101. The ETM 105 may have similar pulse generation circuitry as the IPG 102 to deliver electrical modulation energy to the electrodes in accordance with a set of modulation parameters. The ETM 105 is a non-implantable device that may be used on a trial basis after the neuromodulation leads 101 have been implanted and prior to implantation of the IPG 102, to test the responsiveness of the modulation that is to be provided. Functions described herein with respect to the IPG 102 can likewise be performed with respect to the ETM 105.

The RC 103 may be used to telemetrically control the ETM 105 via a bi-directional RF communications link 109. The RC 103 may be used to telemetrically control the IPG 102 via a bi-directional RF communications link 110. Such control allows the IPG 102 to be turned on or off and to be programmed with different modulation parameter sets. The IPG 102 may also be operated to modify the programmed modulation parameters to actively control the characteristics of the electrical modulation energy output by the IPG 102. A clinician may use the CP 104 to program modulation parameters into the IPG 102 and ETM 105 in the operating room and in follow-up sessions.

The CP 104 may indirectly communicate with the IPG 102 or ETM 105, through the RC 103, via an IR communications link 111 or another link. The CP 104 may directly communicate with the IPG 102 or ETM 105 via an RF communications link or other link (not shown). The clinician detailed modulation parameters provided by the CP 104 may also be used to program the RC 103, so that the modulation parameters can be subsequently modified by operation of the RC 103 in a stand-alone mode (i.e., without the assistance of the CP 104). Various devices may function as the CP 104. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 104. Alternatively, such programming methodologies can be performed using firmware or hardware. In any event, the CP 104 may actively control the characteristics of the electrical modulation generated by the IPG 102 to allow the desired parameters to be determined based on patient feedback or other feedback and for subsequently programming the IPG 102 with the desired modulation parameters. To allow the user to perform these functions, the CP 104 may include user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g., CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical modulation energy output by the neuromodulation leads, and select and program the IPG with modulation parameters, including electrode selection, in both a surgical setting and a clinical setting. The external device(s) (e.g., CP and/or RC) may be configured to communicate with other device(s), including local device(s) and/or remote device(s). For example, wired and/or wireless communication may be used to communicate between or among the devices.

An external charger 112 may be a portable device used to transcutaneous charge the IPG 102 via a wireless link such as an inductive link 113. Once the IPG 102 has been programmed, and its power source has been charged by the external charger or otherwise replenished, the IPG 102 may function as programmed without the RC 103 or CP 104 being present.

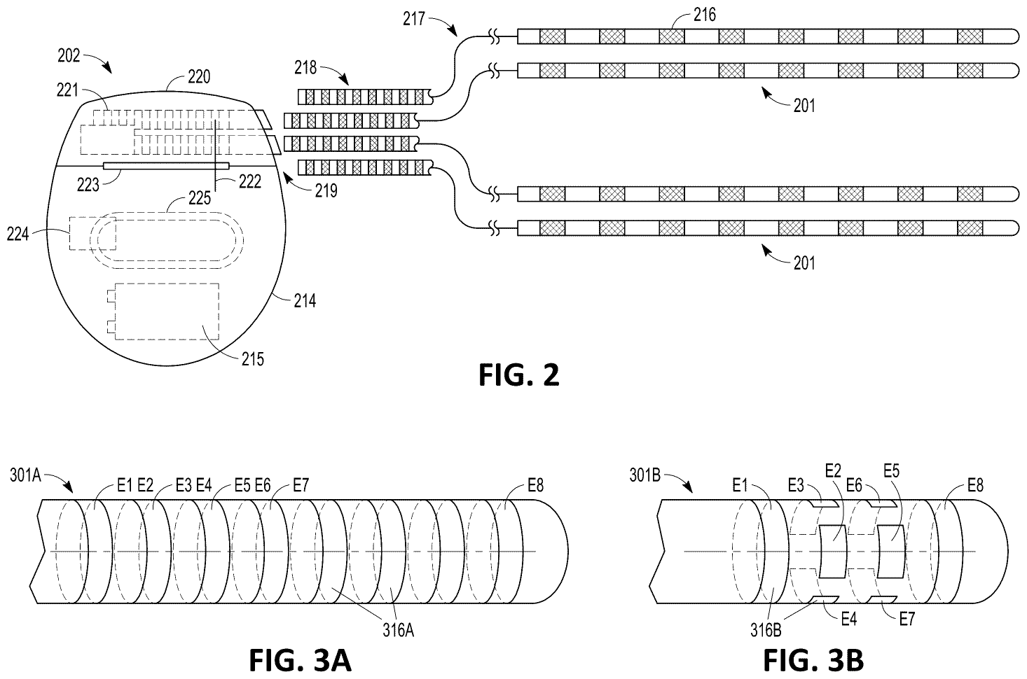

FIG. 2 illustrates, by way of example and not limitation, an IPG 202 in a DBS system. The IPG 202, which is an example of the IPG 102 of the electrical stimulation system 100 as illustrated in FIG. 1, may include a biocompatible device case 214 that holds the circuitry and a battery 215 for providing power for the IPG 202 to function, although the IPG 202 can also lack a battery and can be wirelessly powered by an external source. The IPG 202 may be coupled to one or more leads, such as leads 201 as illustrated herein. The leads 201 can each include a plurality of electrodes 216 for delivering electrostimulation energy, recording electrical signals, or both. In some examples, the leads 201 can be rotatable so that the electrodes 216 can be aligned with the target neurons after the neurons have been located such as based on the recorded signals. The electrodes 216 can include one or more ring electrodes, and/or one or more sets of segmented electrodes (or any other combination of electrodes), examples of which are discussed below with reference to FIGS. 3A and 3B.

The leads 201 can be implanted near or within the desired portion of the body to be stimulated. In an example of operations for DBS, access to the desired position in the brain can be accomplished by drilling a hole in the patient's skull or cranium with a cranial drill (commonly referred to as a burr), and coagulating and incising the dura mater, or brain covering. A lead can then be inserted into the cranium and brain tissue with the assistance of a stylet (not shown). The lead can be guided to the target location within the brain using, for example, a stereotactic frame and a microdrive motor system. In some examples, the microdrive motor system can be fully or partially automatic. The microdrive motor system may be configured to perform actions such as inserting, advancing, rotating, or retracing the lead.

Lead wires 217 within the leads may be coupled to the electrodes 216 and to proximal contacts 218 insertable into lead connectors 219 fixed in a header 220 on the IPG 202, which header can comprise an epoxy for example. Alternatively, the proximal contacts 218 may connect to lead extensions (not shown) which are in turn inserted into the lead connectors 219. Once inserted, the proximal contacts 218 connect to header contacts 221 within the lead connectors 219, which are in turn coupled by feedthrough pins 222 through a case feedthrough 223 to stimulation circuitry 224 within the case 214. The type and number of leads, and the number of electrodes, in an IPG is application specific and therefore can vary.

The IPG 202 can include an antenna 225 allowing it to communicate bi-directionally with a number of external devices. The antenna 225 may be a conductive coil within the case 214, although the coil of the antenna 225 may also appear in the header 220. When the antenna 225 is configured as a coil, communication with external devices may occur using near-field magnetic induction. The IPG 225 may also include a Radio-Frequency (RF) antenna. The RF antenna may comprise a patch, slot, or wire, and may operate as a monopole or dipole, and preferably communicates using far-field electromagnetic waves, and may operate in accordance with any number of known RF communication standards, such as Bluetooth, Zigbee, WiFi, Medical Implant Communication System (MICS), and the like.

In a DBS application, as is useful in the treatment of tremor in Parkinson's disease for example, the IPG 202 is typically implanted under the patient's clavicle (collarbone). The leads 201 (which may be extended by lead extensions, not shown) can be tunneled through and under the neck and the scalp, with the electrodes 216 implanted through holes drilled in the skull and positioned for example in the subthalamic nucleus (STN) and the pedunculopontine nucleus (PPN) in each brain hemisphere. The IPG 202 can also be implanted underneath the scalp closer to the location of the electrodes' implantation. The leads 201, or the extensions, can be integrated with and permanently connected to the IPG 202 in other solutions.

Stimulation in IPG 202 is typically provided by pulses each of which may include one phase or multiple phases. For example, a monopolar stimulation current can be delivered between a lead-based electrode (e.g., one of the electrodes 216) and a case electrode. A bipolar stimulation current can be delivered between two lead-based electrodes (e.g., two of the electrodes 216). Stimulation parameters typically include current amplitude (or voltage amplitude), frequency, pulse width of the pulses or of its individual phases; electrodes selected to provide the stimulation; polarity of such selected electrodes, i.e., whether they act as anodes that source current to the tissue, or cathodes that sink current from the tissue. Each of the electrodes can either be used (an active electrode) or unused (OFF). When the electrode is used, the electrode can be used as an anode or cathode and carry anodic or cathodic current. In some instances, an electrode might be an anode for a period of time and a cathode for a period of time. These and possibly other stimulation parameters taken together comprise a stimulation program that the stimulation circuitry 224 in the IPG 202 can execute to provide therapeutic stimulation to a patient.

In some examples, a measurement device coupled to the muscles or other tissue stimulated by the target neurons, or a unit responsive to the patient or clinician, can be coupled to the IPG 202 or microdrive motor system. The measurement device, user, or clinician can indicate a response by the target muscles or other tissue to the stimulation or recording electrode(s) to further identify the target neurons and facilitate positioning of the stimulation electrode(s). For example, if the target neurons are directed to a muscle experiencing tremors, a measurement device can be used to observe the muscle and indicate changes in, for example, tremor frequency or amplitude in response to stimulation of neurons. Alternatively, the patient or clinician can observe the muscle and provide feedback.

FIGS. 3A-3B illustrate, by way of example and not limitation, leads that may be coupled to the IPG to deliver electrostimulation such as DBS. FIG. 3A shows a lead 301A with electrodes 316A disposed at least partially about a circumference of the lead 301A. The electrodes 316A may be located along a distal end portion of the lead. As illustrated herein, the electrodes 316A are ring electrodes that span 360 degrees about a circumference of the lead 301. A ring electrode allows current to project equally in every direction from the position of the electrode, and typically does not enable stimulus current to be directed from only a particular angular position or a limited angular range around of the lead. A lead which includes only ring electrodes may be referred to as a non-directional lead.

FIG. 3B shows a lead 301B with electrodes 316B including ring electrodes such as E1 at a proximal end and E8 at the distal end. Additionally, the lead 301 also include a plurality of segmented electrodes (also known as split-ring electrodes). For example, a set of segmented electrodes E2, E3, and E4 are around the circumference at a longitudinal position, each spanning less than 360 degrees around the lead axis. In an example, each of electrodes E2, E3, and E4 spans 90 degrees, with each being separated from the others by gaps of 30 degrees. Another set of segmented electrodes E5, E6, and E7 are located around the circumference at another longitudinal position different from the segmented electrodes E2, E3 and E4. Segmented electrodes such as E2-E7 can direct stimulus current to a selected angular range around the lead.

Segmented electrodes can typically provide superior current steering than ring electrodes because target structures in DBS or other stimulation are not typically symmetric about the axis of the distal electrode array. Instead, a target may be located on one side of a plane running through the axis of the lead. Through the use of a radially segmented electrode array, current steering can be performed not only along a length of the lead but also around a circumference of the lead. This provides precise three-dimensional targeting and delivery of the current stimulus to neural target tissue, while potentially avoiding stimulation of other tissue. In some examples, segmented electrodes can be together with ring electrodes. A lead which includes at least one or more segmented electrodes may be referred to as a directional lead. In an example, all electrodes on a directional lead can be segmented electrodes. In another example, there can be different numbers of segmented electrodes at different longitudinal positions.

Segmented electrodes may be grouped into sets of segmented electrodes, where each set is disposed around a circumference at a particular longitudinal location of the directional lead. The directional lead may have any number of segmented electrodes in a given set of segmented electrodes. By way of example and not limitation, a given set may include any number between two to sixteen segmented electrodes. In an example, all sets of segmented electrodes may contain the same number of segmented electrodes. In another example, one set of the segmented electrodes may include a different number of electrodes than at least one other set of segmented electrodes.

The segmented electrodes may vary in size and shape. In some examples, the segmented electrodes are all of the same size, shape, diameter, width or area or any combination thereof. In some examples, the segmented electrodes of each circumferential set (or even all segmented electrodes disposed on the lead) may be identical in size and shape. The sets of segmented electrodes may be positioned in irregular or regular intervals along a length the lead 219.

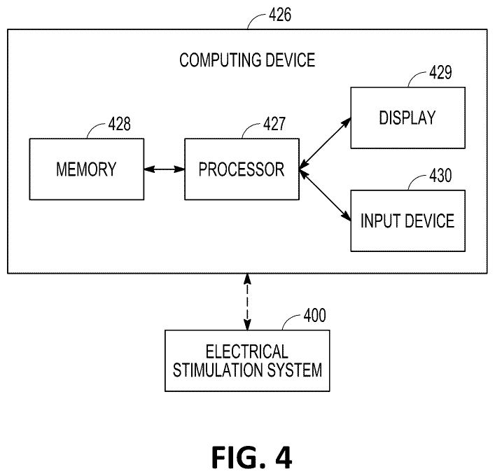

FIG. 4 illustrates, by way of example and not limitation, a computing device 426 for programming or controlling the operation of an electrical stimulation system 400. The computing device 426 may include a processor 427, a memory 428, a display 429, and an input device 430. Optionally, the computing device 426 may be separate from and communicatively coupled to the electrical stimulation system 400, such as system 100 in FIG. 1. Alternatively, the computing device 426 may be integrated with the electrical stimulation system 100, such as part of the IPG 102, RC 103, CP 104, or ETM 105 illustrated in FIG. 1. The computing device may be used to perform process(s) for sensing parameter(s).

The computing device 426, also referred to as a programming device, can be a computer, tablet, mobile device, or any other suitable device for processing information. The computing device 426 can be local to the user or can include components that are non-local to the computer including one or both of the processor 427 or memory 428 (or portions thereof). For example, the user may operate a terminal that is connected to a non-local processor or memory. The functions associated with the computing device 426 may be distributed among two or more devices, such that there may be two or more memory devices performing memory functions, two or more processors performing processing functions, two or more displays performing display functions, and/or two or more input devices performing input functions. In some examples, the computing device 406 can include a watch, wristband, smartphone, or the like. Such computing devices can wirelessly communicate with the other components of the electrical stimulation system, such as the CP 104, RC 103, ETM 105, or IPG 102 illustrated in FIG. 1. The computing device 426 may be used for gathering patient information, such as general activity level or present queries or tests to the patient to identify or score pain, depression, stimulation effects or side effects, cognitive ability, or the like. In some examples, the computing device 426 may prompt the patient to take a periodic test (for example, every day) for cognitive ability to monitor, for example, Alzheimer's disease. In some examples, the computing device 426 may detect, or otherwise receive as input, patient clinical responses to electrostimulation such as DBS, and determine or update stimulation parameters using a closed- loop algorithm based on the patient clinical responses. Examples of the patient clinical responses may include physiological signals (e.g., heart rate) or motor parameters (e.g., tremor, rigidity, bradykinesia). The computing device 426 may communicate with the CP 104, RC 103, ETM 105, or IPG 102 and direct the changes to the stimulation parameters to one or more of those devices. In some examples, the computing device 426 can be a wearable device used by the patient only during programming sessions. Alternatively, the computing device 426 can be worn all the time and continually or periodically adjust the stimulation parameters. In an example, a closed-loop algorithm for determining or updating stimulation parameters can be implemented in a mobile device, such as a smartphone, that is connected to the IPG or an evaluating device (e.g., a wristband or watch). These devices can also record and send information to the clinician.

The processor 427 may include one or more processors that may be local to the user or non-local to the user or other components of the computing device 426. A stimulation setting (e.g., parameter set) includes an electrode configuration and values for one or more stimulation parameters. The electrode configuration may include information about electrodes (ring electrodes and/or segmented electrodes) selected to be active for delivering stimulation (ON) or inactive (OFF), polarity of the selected electrodes, electrode locations (e.g., longitudinal positions of ring electrodes along the length of a non-directional lead, or longitudinal positions and angular positions of segmented electrodes on a circumference at a longitudinal position of a directional lead), stimulation modes such as monopolar pacing or bipolar pacing, etc. The stimulation parameters may include, for example, current amplitude values, current fractionalization across electrodes, stimulation frequency, stimulation pulse width, etc.

The processor 427 may identify or modify a stimulation setting through an optimization process until a search criterion is satisfied, such as until an optimal, desired, or acceptable patient clinical response is achieved. Electrostimulation programmed with a setting may be delivered to the patient, clinical effects (including therapeutic effects and/or side effects, or motor symptoms such as bradykinesia, tremor, or rigidity) may be detected, and a clinical response may be evaluated based on the detected clinical effects. When actual electrostimulation is administered, the settings may be referred to as tested settings, and the clinical responses may be referred to as tested clinical responses. In contrast, for a setting in which no electrostimulation is delivered to the patient, clinical effects may be predicted using a computational model based at least on the clinical effects detected from the tested settings, and a clinical response may be estimated using the predicted clinical effects. When no electrostimulation is delivered the settings may be referred to as predicted or estimated settings, and the clinical responses may be referred to as predicted or estimated clinical responses.

In various examples, portions of the functions of the processor 427 may be implemented as a part of a microprocessor circuit. The microprocessor circuit can be a dedicated processor such as a digital signal processor, application specific integrated circuit (ASIC), microprocessor, or other type of processor for processing information. Alternatively, the microprocessor circuit can be a processor that can receive and execute a set of instructions of performing the functions, methods, or techniques described herein.

The memory 428 can store instructions executable by the processor 427 to perform various functions including, for example, determining a reduced or restricted electrode configuration and parameter search space (also referred to as a “restricted search space”), creating or modifying one or more stimulation settings within the restricted search space, etc. The memory 428 may store the search space, the stimulation settings including the “tested” stimulation settings and the “predicted” or “estimated” stimulation settings, clinical effects (e.g., therapeutic effects and/or side effects) and clinical responses for the settings.

The memory 428 may be a computer-readable storage media that includes, for example, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information, and which can be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, Bluetooth, near field communication, and other wireless media.

The display 429 may be any suitable display or presentation device, such as a monitor, screen, display, or the like, and can include a printer. The display 429 may be a part of a user interface configured to display information about stimulation settings (e.g., electrode configurations and stimulation parameter values and value ranges) and user control elements for programming a stimulation setting into an IPG. The computing device 426 may include other output(s) such as speaker(s) and haptic output(s) (e.g., vibration motor).

The input device 430 may be, for example, a keyboard, mouse, touch screen, track ball, joystick, voice recognition system, or any combination thereof, or the like. Another input device 430 may be a camera from which the clinician can observe the patient. Yet another input device 430 may a microphone where the patient or clinician can provide responses or queries.

The electrical stimulation system 400 may include, for example, any of the components illustrated in FIG. 1. The electrical stimulation system 400 may communicate with the computing device 426 through a wired or wireless connection or, alternatively or additionally, a user can provide information between the electrical stimulation system 400 and the computing device 426 using a computer-readable medium or by some other mechanism.

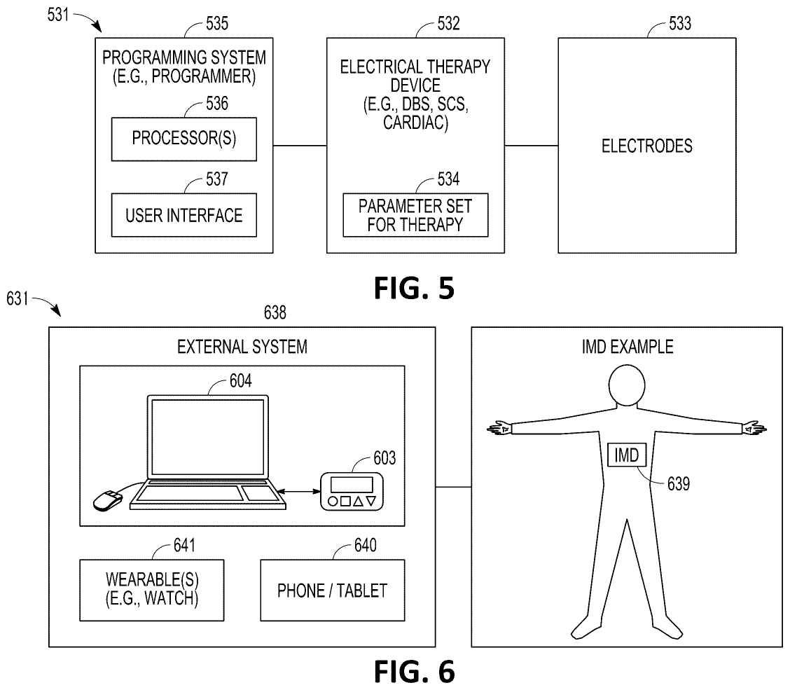

FIG. 5 illustrates, by way of example, an example of an electrical therapy-delivery system. The illustrated system 531 includes an electrical therapy device 532 configured to deliver an electrical therapy to electrodes 533 to treat a condition in accordance with a programmed parameter set 534 for the therapy. The system 531 may include a programming system 535, which may function as at least a portion of a processing system, that may include one or more processors 536 and a user interface 537. The programming system 535 may be used to program and/or evaluate the parameter set(s) used to deliver the therapy. The illustrated system 531 may be a DBS system.

In some embodiments, the illustrated system 531 may include an SCS system to treat pain and/or a system for monitoring pain. By way of example, a therapeutic goal for conventional SCS programming may be to maximize stimulation (i.e., recruitment) of the dorsal column (DC) fibers that run in the white matter along the longitudinal axis of the spinal cord and minimal stimulation of other fibers that run perpendicular to the longitudinal axis of the spinal cord (e.g., dorsal root fibers).

A therapy may be delivered according to a parameter set. The parameter set may be programmed into the device to deliver the specific therapy using specific values for a plurality of therapy parameters. For example, the therapy parameters that control the therapy may include pulse amplitude, pulse frequency, pulse width, and electrode configuration (e.g., selected electrodes, polarity and fractionalization). The parameter set includes specific values for the therapy parameters. The number of electrodes available combined with the ability to generate a variety of complex electrical waveforms (e.g., pulses), presents a huge selection of modulation parameter sets to the clinician or patient. For example, if the neuromodulation system to be programmed has sixteen electrodes, millions of modulation parameter sets may be available for programming into the neuromodulation system. To facilitate such selection, the clinician generally programs the modulation parameters sets through a computerized programming system to allow the optimum modulation parameters to be determined based on patient feedback or other means and to subsequently program the desired modulation parameter sets.

FIG. 6 illustrates, by way of example and not limitation, the electrical therapy- delivery system of FIG. 5 implemented using an IMD. The illustrated system 631 includes an external system 638 that may include at least one programming device. The illustrated external system 638 may include a clinician programmer 604, similar to CP 104 in FIG. 1, configured for use by a clinician to communicate with and program the neuromodulator, and a remote control device 603, similar to RC 103 in FIG. 1, configured for use by the patient to communicate with and program the neuromodulator. For example, the remote control device 603 may allow the patient to turn a therapy on and off, change or select programs, and/or may allow the patient to adjust patient-programmable parameter(s) of the plurality of modulation parameters. FIG. 6 illustrates an IMD 639, although the monitor and/or therapy device may be an external device such as a wearable device. The external system 638 may include a network of computers, including computer(s) remotely located from the IMD 639 that are capable of communicating via one or more communication networks with the programmer 604 and/or the remote control device 603. The remotely located computer(s) and the IMD 639 may be configured to communicate with each other via another external device such as the programmer 604 or the remote control device 603. The remote control device 603 and/or the programmer 604 may allow a user (e.g., patient and/or clinician or rep) to answer questions as part of a data collection process. The external system 638 may include personal devices such as a phone or tablet 640, wearables such as a watch 641, sensors or therapy-applying devices. The watch may include sensor(s), such as sensor(s) for detecting activity, motion and/or posture. Other wearable sensor(s) may be configured for use to detect activity, motion and/or posture of the patient. The external system 638 may include, but is not limited to, a phone and/or a tablet. Notifications may be sent to the patient, physician, device rep or other users via the external system and through remote portals (e.g., web-based portals) provided by remote systems.

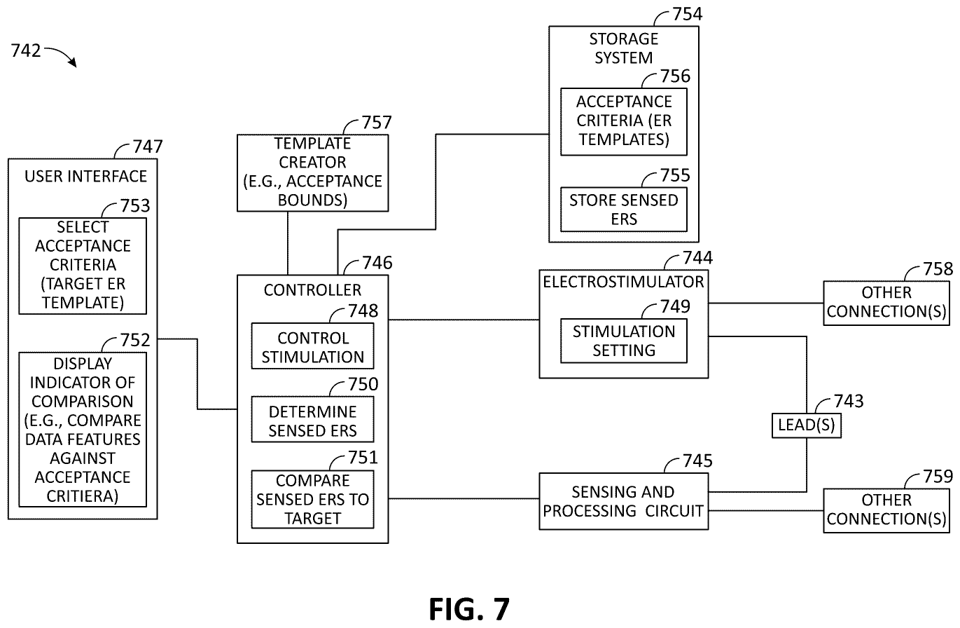

FIG. 7 illustrates a system for comparing sensed ERs to electrical stimulation against acceptance criteria. The illustrated system 742 includes at least one lead 743 with electrodes, an electrostimulator 744 operably connected to the lead(s), a sensing circuit 745 operably connected to the lead(s), a controller 746 and a user interface 747. The controller 746 is connected to the electrostimulator 744 and is configured to provide stimulation control 748. The electrostimulator 744 may be configured to deliver electrical stimulation according to a stimulation setting 749. The stimulation setting 749 may include parameters such as, but not limited to, pulse amplitude pulse width pulse frequency and fractionalization. For example, the controller may program the stimulation settings 749 into the electrostimulator 744 and may control timing for delivering pulse waves. The controller 746 is also connected to the sensing and processing circuit 745 and is configured to determine sensed ERs 750 and compare the sensed ERs to a target 751. The user interface 747 may be operably connected to the controller 746 and may be configured to provide a display of an indicator of the comparison of the sense ERS to the target or acceptance criteria 752. The user interface 747 may also be configured to receive an input from a user regarding the acceptance criteria 753. For example, the user may select a template for the desired ERs. The user interface 747 may also receive from the user acceptance bounds for the sensed ERs. The illustrated system 742 may include a storage system 754 configured to store sensed ERs 755 and, in some embodiments, to store acceptance criteria for the ERs 756. Some system embodiments may include a template creator 757. By way of example and not limitation, the template creator 757 may be configured to use machine learning to create ER templates for selection by the user.

Machine learning (also referred to as Artificial Intelligence or AI) may be implemented on patient-specific and/or patient population data to refine and improve upon the therapeutic response to various inputs. The training data may be used by a machine learning algorithm to determine relationship(s) between the sensed electrical activity (e.g., extracted feature(s) of an electrical signal) and lead placements and/or the parameter(s) of the neuromodulation. The method may include, at 308, performing a training procedure to determine a relationship between sensed electrical activity and neuromodulation parameters. Examples of sensed electrical activity include neural activity or muscle activity. Examples include local field potentials, evoked compound action potentials (ECAPs), or evoked resonant neural activity (ERNA). The system may be design to use various different ways to train and deploy, including train/update/refine on CP, or in cloud, using various levels of localized/partitioned data such as using only some of my patients, using my patients and patients from select other providers, everyone with my target (+ methods), and everyone in the country.

The ER templates may be desired distributions or ranges or other criteria of ERs. The electrical stimulator may be connected with other connection(s) 758 in the sensing and processing circuit may be connected with other connection(s) 759. This disclosure refers to DBS systems as an example in which different user-defined ER targets for stimulation, and further refers to ERNA as an example of an ER. The present subject matter may be used with other DBS evoked potentials, other DBS spontaneous potentials such as local field potentials (LFPs), and other indications and their evoked potentials, such as spinal cord stimulation (SCS) and peripheral nerve stimulation (PNS) (e.g., vagal nerve stimulation (VNS), sacral nerve stimulation (SNS), occipital nerve stimulation (ONS), and the like). A target may match particular data or features of data, collections (distributions) of features, and may use tolerances or bounds. A target may relate to a location of a feature (or of a distribution of features) in space. A target may relate to a change of a feature across a sweep with a varying parameter. By way of example and not limitation, the system may look for +10% response when delivering +10% amplitude, or that rate feature decays by half after evoking for 30s, and the like.

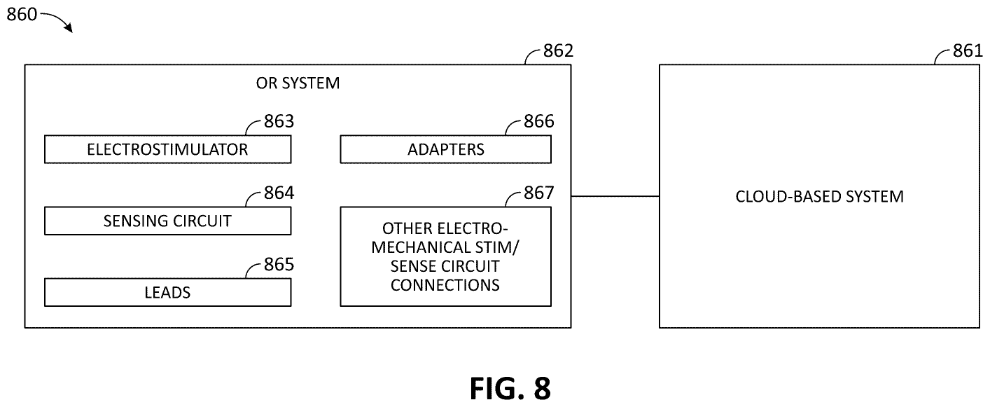

FIG. 8 illustrates the system that may be used to implement the present subject matter. The illustrated system 860 includes a cloud-based system 861 which may include one or more cloud-based applications. The system 860 may also include an operating room system 862 that is configured to be used within an operating room during an implantation procedure. The operating room system 862 may include an electrostimulator 863 such as deep brain stimulator for implantation in the patient. The operating room system 862 may further include a sensing circuit 864 configured to be used to sense the ERs. Both the electrostimulator 863 and the sensing circuit 864 may be connected to a lead 865 or more than one lead such as a deep brain stimulation lead. The operating room system 862 may further include adapters 866 and other electromechanical stimulations and sensing circuit connections 867. The cloud-based system 861 and/or the operating room system 862 may be configured to provide the ER targets that may be compared against the sensed ERs. It is noted that the device(s) used to provide stimulation and perform sensing that causes the ERs may include a device used in the operating room that is not a DBS stimulator, a DBS stimulator used in the operating room, or an implanted DBS stimulator used outside of operating room (clinic, general environment)

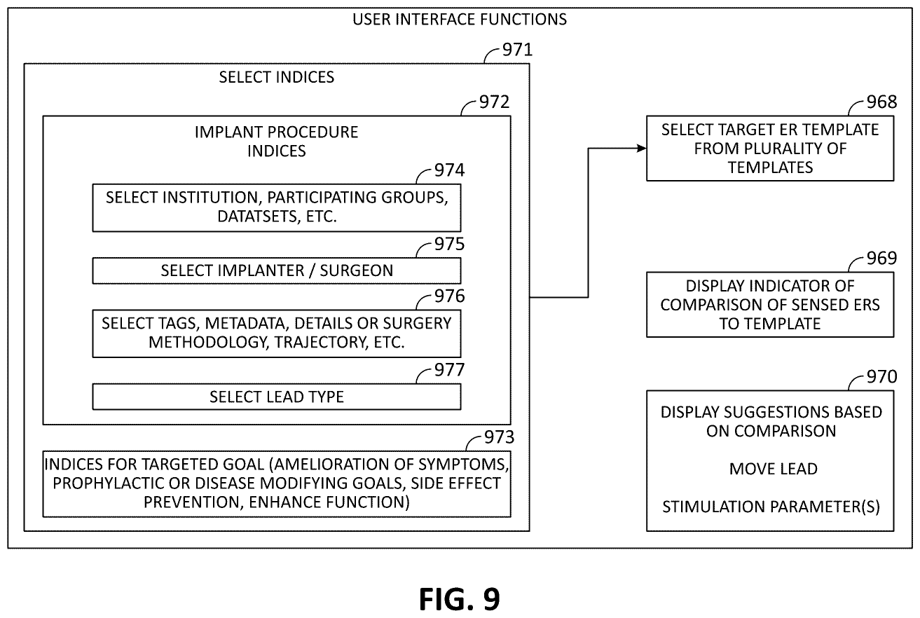

FIG. 9 illustrates, by way of example and not limitation, some user interface functionality according to various embodiments of the present subject matter. The user interface may be used to select the target ER 746 and template from a plurality of templates 968. The sensed ERs may be compared against the selected target. The user interface may be configured to display an indicator of the comparison of the sense ERs to the template 969. In some embodiments, the user interface is configured to display suggestions based on the comparison 970. For example, during an implantation procedure, the user may suggest moving the lead to cause the ERs to align or more favorably compare to the template or other acceptable criteria. In some embodiments the display may provide a suggestion to the user to adjust stimulation parameters to cause the since developed responses to more favorably compared to the ER template.

In some embodiments the selection of the target ER template may be based on a selected index or indices 971. By way of example and not limitation the indices may be selected based on an implant procedure 972 or may be selected based on a targeted goal 973. Implant procedure indices 972 may include a select institution, participating group, datasets etc. 974. Implant procedure indices 972 may include a selected implanter or surgeon 975. Implant procedure indices 972 may include selected tags, metadata, details of surgery or methodology, trajectory of lead implant, and the like 976. Implant procedure indices 972 may include a lead type 977. The indices for targeted goal 973 may include amelioration of symptoms, prophylactic or disease modifying goals, side effect prevention, or enhance function. For example, the target ER template may be selected to relieve symptoms or for other goals such as lead placement for disease-modifying therapy or co-therapy (e.g., leads that inject drugs or light), and side-effect avoidance.

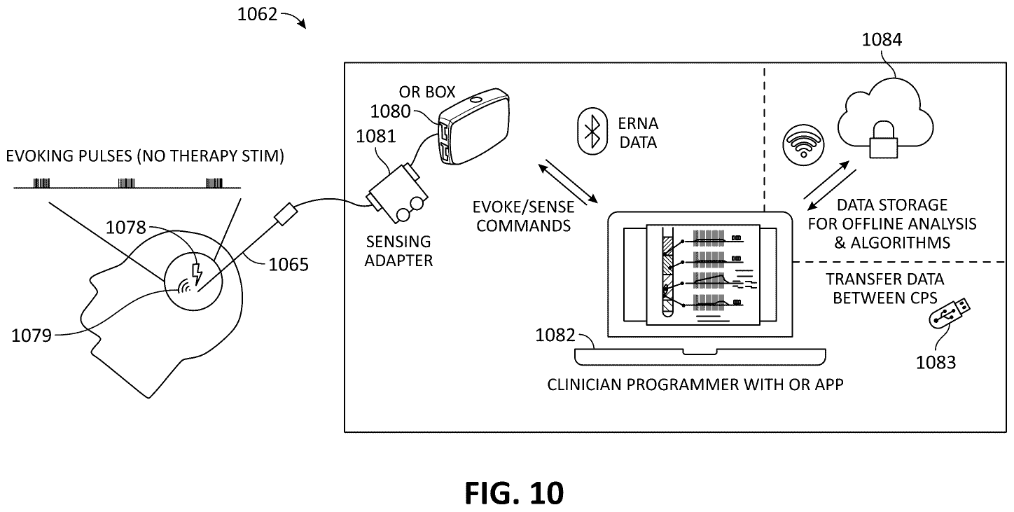

FIG. 10 illustrates, by way of example and not limitation, an operating room system 1062. The OR system 1062 includes a DBS lead 1065 configured to deliver neurostimulation 1078 to brain tissue and to detect ERs (e.g., evoked pulses) 1079 to the delivered neurostimulation. An OR box 1080 and sensing adaptor 1081 may be used to deliver the neurostimulation 1078 and/or detect ERs 1079 using the DBS lead 1065. The OR box 1080 may be configured to communicate with a clinician programmer 1082, where the communication may include evoke commands used to generate the neurostimulation and may include sensing commands to sensed response data such as ERNA data. The clinician programmer 1082 may be configured with an Operating Room (OR) app used to assist with monitoring ERs during lead placement and/or neurostimulation programming. The response data (e.g., ERNA) may be extracted, processed, stored and analyzed using other systems such as other clinician programmers via network communication or portable memory 1083 or cloud-based storage/applications 1084. Application(s) such as software in the OR app or the cloud-based applications) may be used to the fingerprints (e.g., ER target(s)) to be used in a center. The ER target(s) may be pushed to the OR box to determine if the ER(s) sensed from the patient matches the ER target(s)/fingerprint(s). The OR app may be deployed in different institutions and used by different researchers. These different researchers my coordinate their work to create some ER targets together. For example, individually-collected data from the patient may be pushed to the cloud and shared by others. The storage (e.g., cloud-based storage) may include a library or database storing ER signals of patients, neuroanatomies, system information, target(s). Cloud-based analysis may be performed, and the processing results may be pushed down to OR apps and shared by users.

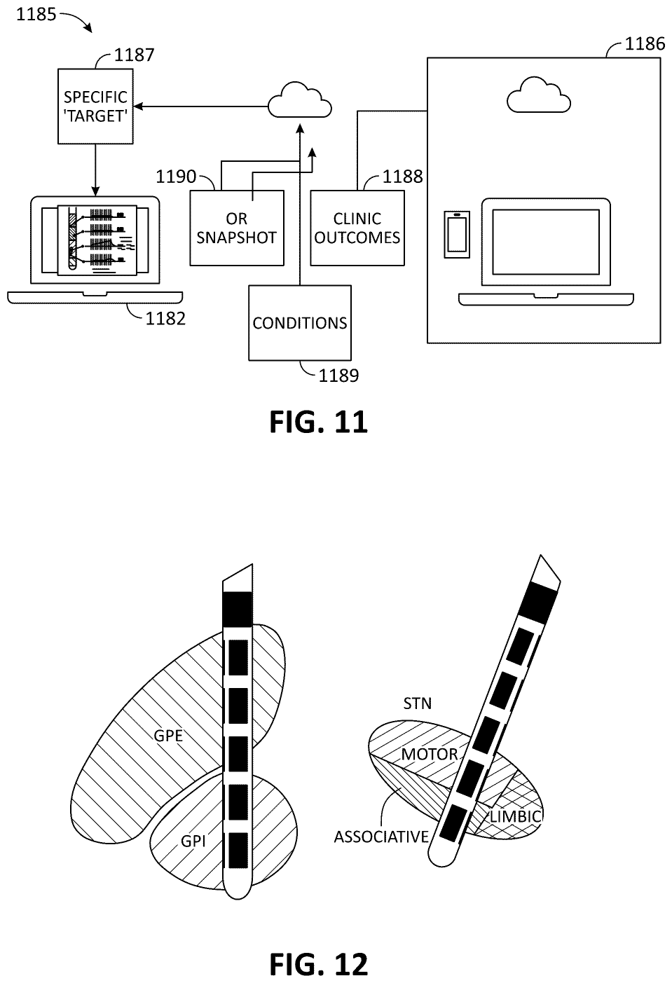

FIG. 11 illustrates a system configured for use to compare ER data to a specific target response, and to update the target response based on a number of factors. The illustrated system 1185 may include a clinician programmer 1182 and other systems (e.g., cloud-based applications, tablets, phones or other clinician programmer) 1186. The factors for determine ER target(s) 1187 may include additional data, other than ERs, such as clinical outcomes 1188, conditions 1189 and operating room specifics (“OR snapshot”) 1190 such as anesthesia. For example, the factors may include patient condition, anatomy and trajectory of the anatomical target. The target response may include thresholds, shapes, trained classifiers, and the like. Examples of conditions may include evoking parameters, patient condition (diagnosis, disease duration, symptoms, meds) target (anatomy, trajectory), OR specifics (anesthesia). Outcomes may include, by way of example and not limitation, a physician (implanter) scores whether they like the lead position, a clinician scores whether they like the therapeutic outcome after one or more visits, objective (e.g., wearable sensors) scores at time interval(s) (e.g., 3 mo.) after implant, and patient scores satisfaction of therapy.

It has been proposed to deliver DBS to the subthalamic nucleus (STN), internal global pallidus (GPi) and the external global pallidus (Gpe) for Parkinson's Disease. Other targets and indications are addressed by DBS. FIG. 12 illustrates by way of example and not limitation ERs when the GPe, GPi and STN is stimulated. The ER may be compared to a target response for a given stimulation target to determine when the lead has been properly implanted and/or the program settings have been properly configured.

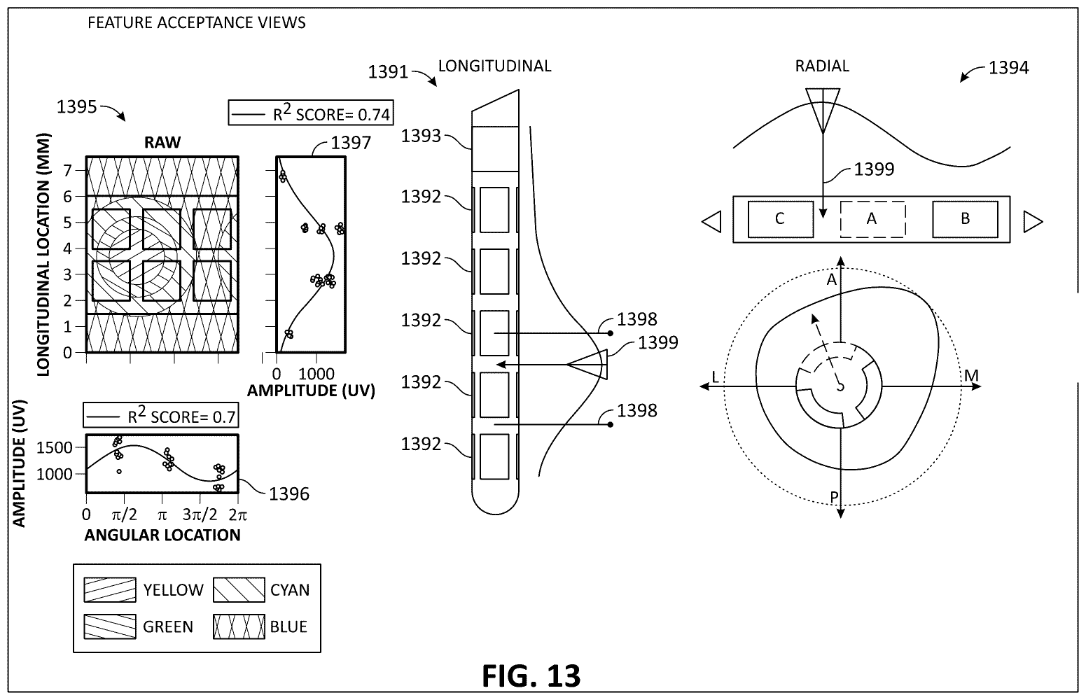

FIG. 13 illustrates, by way of example and not limitation, a display for user interface that creates a response fit from a distribution of ERs. The figure illustrates a longitudinal view of lead 1391 including rows of segmented electrodes 1392 and a ring electrode 1393. The segmented electrodes 1392 are illustrated as three electrode per row (see electrodes a, b and c in the radial view 1394). The present subject matter is not limited to this particular example of a lead with this particular arrangement of the electrodes. A hotspot view 1395 provides an indicator (e.g., color heatmap) for a hotpot for the distribution of ERs, along with a directional depiction 1396 of the distribution of evoked potentials around the segmented electrodes, and a longitudinal depiction 1397 of the distribution of evoked potentials long the longitudinal position of the lead. In FIG. 13, and elsewhere in this application, different patterns are illustrated in the figures to represent different color regions. Fewer or more colors may be used. The color heat map may include color gradations. The hotspot view may be layered over an electrode template representing electrodes on the lead. The distribution depictions 1396 and 1397 may include a best fit line for plotted values for the sensed ERs. Various embodiments may enable the user to place bounds around the desired target for the distribution, such as the 1398 bounds on the longitudinal view of the lead. Arrows 1399 or some other indicator may provide a means to identify the largest distribution along the longitudinal lead and the along the segmented electrodes. A user may choose to accept the lead position and/or stimulation setting based on the displayed images on this display (e.g., based on an arrow 1399 being within the bounds 1398).

Various embodiments provide a user interface with data view that allow a user to set an ER target, sense an ER, and provide an indicator (e.g., highlight) whether a given sensed response matches the ER target set by the user. The sensed response target may include a plurality of sensed response values from a plurality of electrodes which may form a distribution of sensed response values.

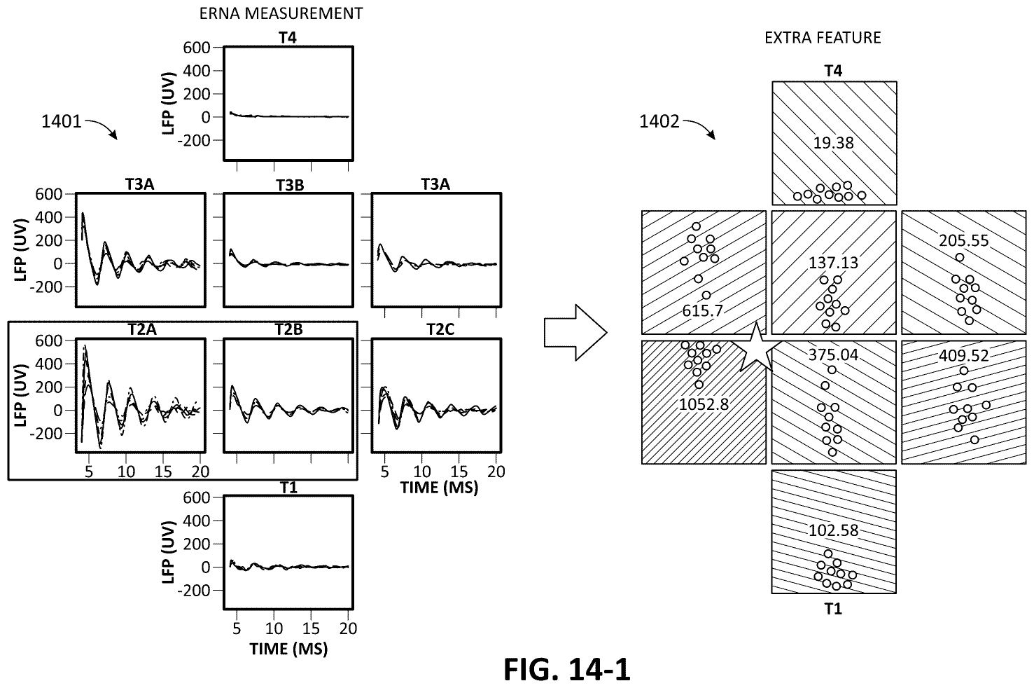

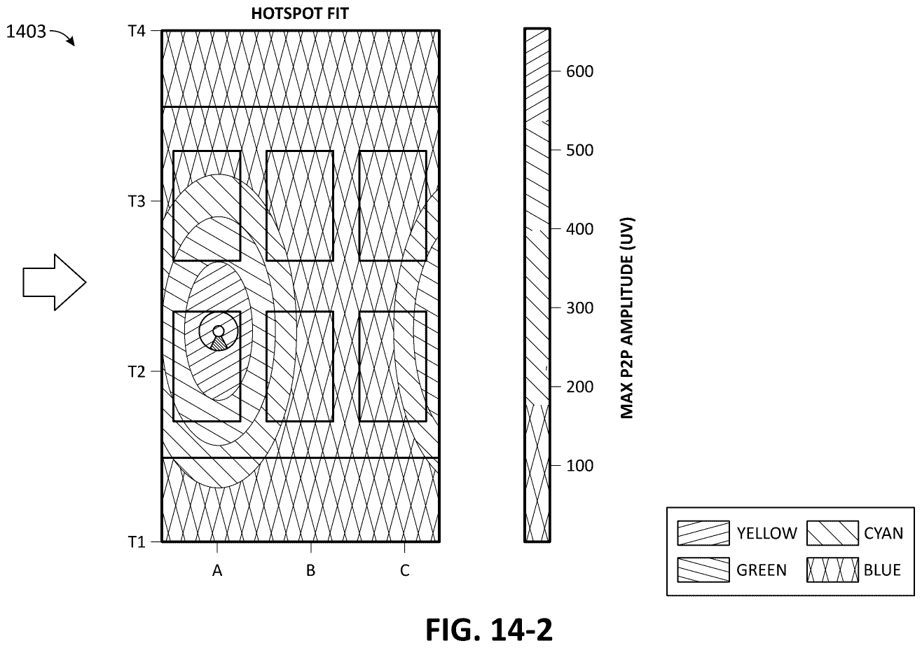

FIGS. 14-1 and 14-2 illustrate, by way of example and not limitation, embodiments for measuring and matching ERs to acceptance criteria such as a template. Each square in the ERN measurement and the extracted feature views represents an electrode (or electrode segment for the segmented electrodes for the directional lead). The figure illustrates, by way of example and not limitation, two rows of segmented electrodes. The raw ERNA measurement is illustrated in the figure at 1401. The ER measurements may be measured at different locations/electrode vertical locations, and directional locations (a, b, c) at a fixed vertical location (e.g., T3a, T3b, T3c). Further multiple measurements may be taken from each set of one or more electrodes. Multiple measurements from each of the locations.

Feature(s) may be extracted from the raw ERNA measurement data and presented in an extracted feature view 1402 that provide a distribution of extracted feature(s). For example, features such as amplitude, magnitude, first peak, width, RMS value, and the like may be extracted from the raw ER signals.

The extracted features may be used to create sweet spot map or a hotspot fit 1403. The hotpot fit may be color coded similar to thermal mapping, where more features result in a “hotter” color. The hotspot may correspond to a peak of the evoked response (e.g., ERNA) distribution. The measured hotspot may be displayed to the user, as well as an indicator (e.g., highlight) whether the measured response matches the user's or center's ER target. For example, an overlay of the measured ER data (feature distribution), or hotspot, with a visual box of the desired hotspot (i.e., the footprint/template for the institution). A match indicates that the correct location is being stimulation. If there is no match, the ER target provides guidance for moving the electrode or adjusting the stimulation setting until the measured ER (sensed distribution) matches the ER target (target distribution). The desired hotspot or target ER distribution may be dependent on the symptom to be treated (e.g., cognitive or motor functions). For example, an ER target for tremor may be near the STN, and an ER target for bradykinesia may be located higher on the need. The ER target may be selected from a database (e.g., library) of available targets to provide acceptable distributions in different directions. Some manners of evoking and recording ER may not lend to visualization of the ‘hotspot’ in a 2d map. This does not preclude the use of the art described herein.

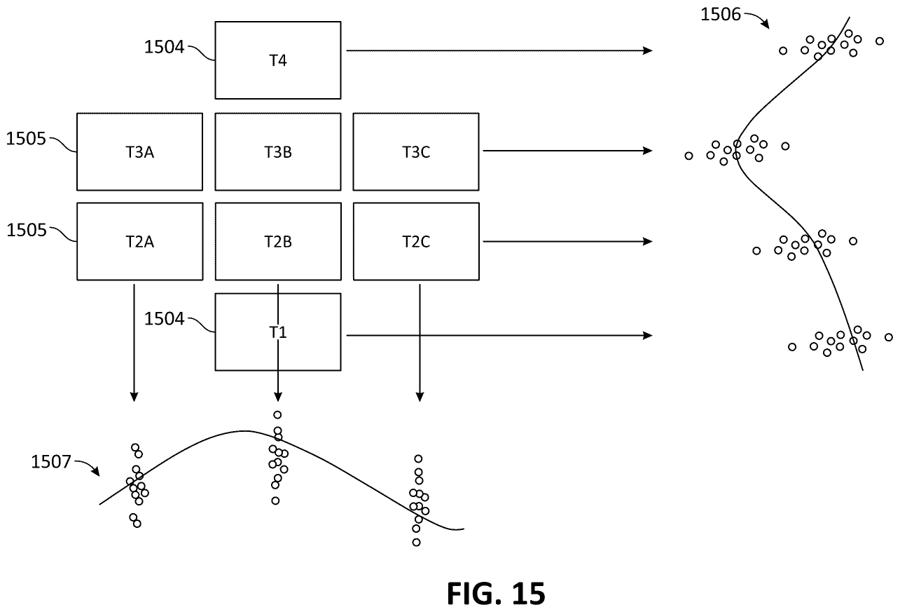

FIG. 15 illustrates, by way of example and not limitation, ER distributions for DBS lead electrodes. The lead may include two ring electrodes 1504 and two segmented electrodes 1505 where each segmented electrode includes three electrodes. The sensed ERs at each electrode are plotted for both the vertical and directional directions. A best fit curve may be created for both the vertical distribution 1506 and directional distribution 1507. The ER target may include at least a portion of a desired best fit curve in the vertical and/or directional distributions. That is, for example, the best fit curve for the sensed ERs may be compared to the best fit curve for the ER target.

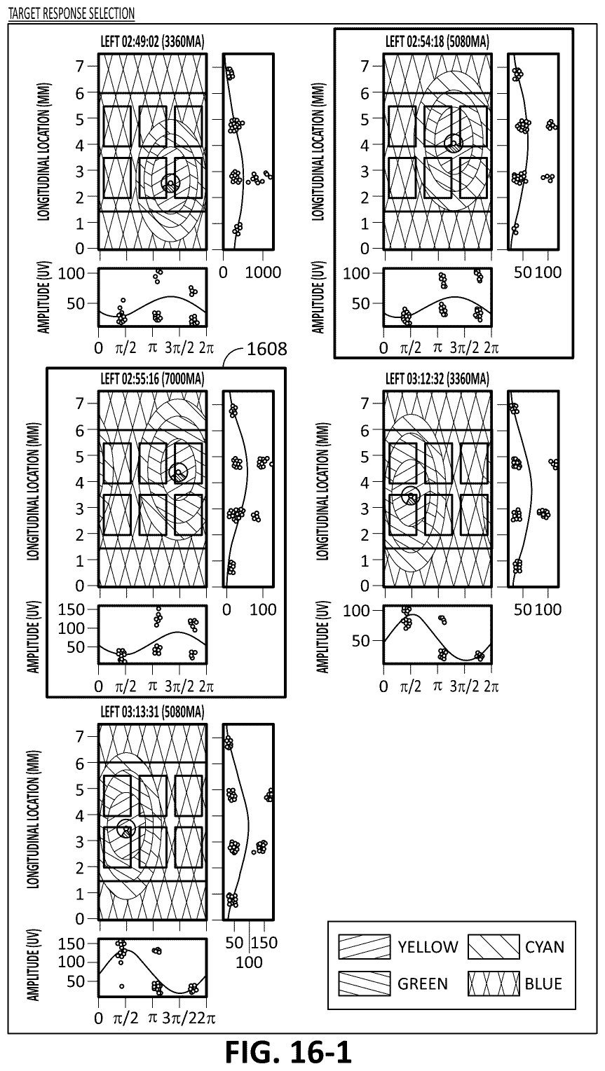

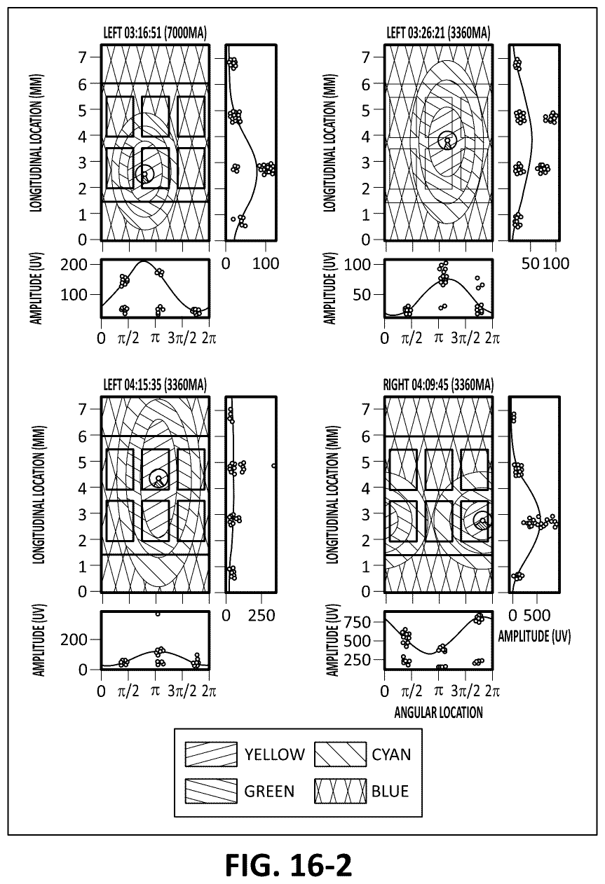

FIGS. 16-1 and 16-2 illustrate, by way of example and not limitation, a library for ER targets. Each ER target 1608 (typical) available for selection may include are representation of a hotspot on a representation (e.g., 2-dimensional representation) of the lead, and may further includes a representation of a desirable distribution (e.g., curve or other representation of the distribution or characteristics of the distribution) for both the vertical and directional dimensions of the lead. Indices may be used to assist with selecting a desired ER target. For example, a user may select an ER target by selecting one or more indices such as a selected surgery center/institution, implanter, tags, metadata, details of surgery or methodology, trajectory of lead implant, symptoms, prophylactic or disease modifying goals, side effect prevention, or function enhancement. For example, the ER target may be selected to relieve symptoms or for other goals such as lead placement for disease-modifying therapy or co-therapy (e.g., leads that inject drugs or light), and side-effect avoidance. The target may be compared to sensed ERs to determine if the lead is properly placed and/or determine a proper stimulation setting.

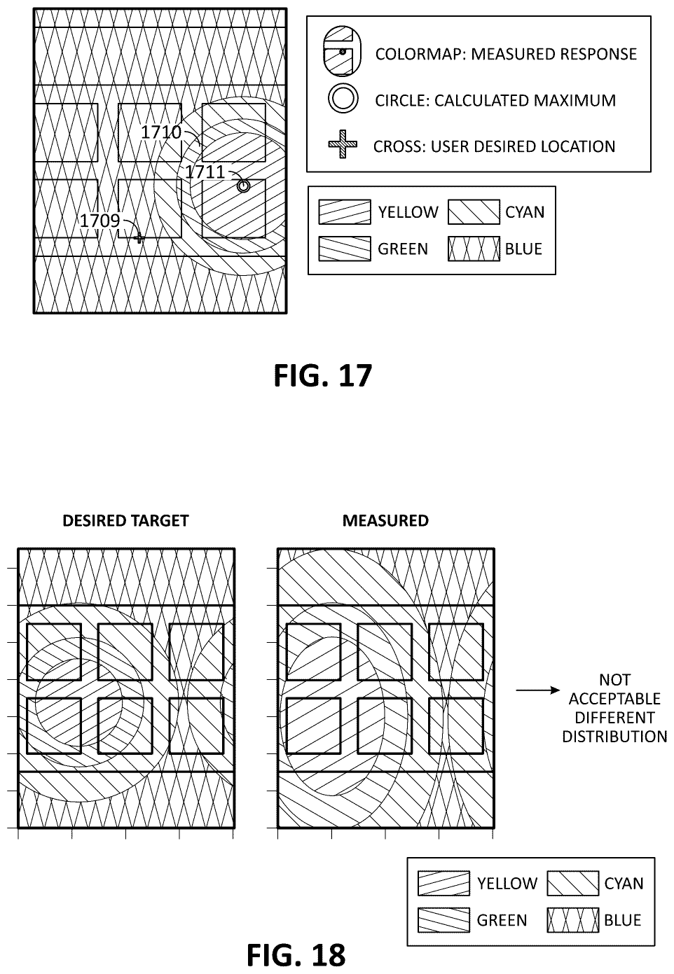

FIG. 17 illustrates, by way of example and not limitation, an example of a view on a user interface for identifying a user-desired location for the sensed response target 1709, the distribution of the sensed ERs 1710, and a calculated 1711 maximum within the distribution of sensed ERs. The lead may be moved and/or the parameter settings may be changed so that the calculated maximum is near the user desired location, and/or to control the distribution “spread” for the sensed ERs.

FIG. 18 illustrates, by way of example and not limitation, an ER target distribution and a measured or sensed ER distribution. This figure illustrates a situation in which the peak location of the sensed or measured ER distribution generally matches the peak in the ER target distribution. However, the “spread” of the peak may be considered to be too broad, as the peak in the ER target distribution is more focused. Therefore, the system may be configured to reject sensed ER distribution from being a match, and may suggest changes to the lead position and/or stimulation settings to further focus the sensed ER distribution.

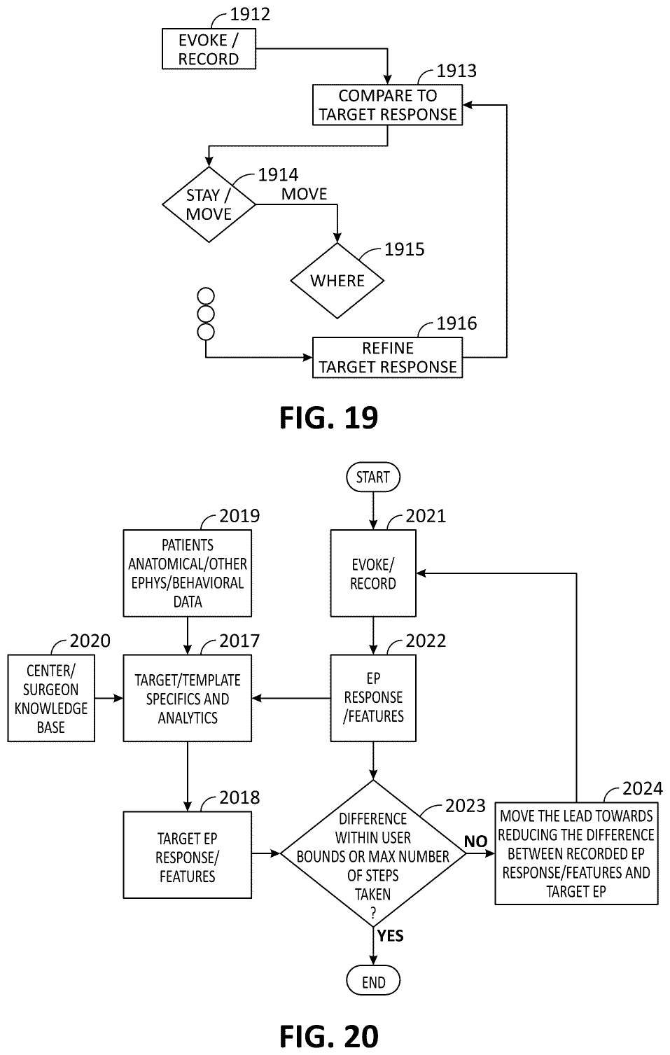

FIG. 19 illustrates, by way of example and not limitation, a method for using sensed ERs. At 1912, the method senses and records the ERs (e.g., ERNA) to the stimulation. This may involve recording many sensed multiple sensed values for each of a plurality of electrodes on the lead(s). The recorded ERs may be compared to an ER target at 1913, which may involve comparing a distribution of sensed values or one or more characteristics of the sensed values or their distribution to the target. The results of the comparison may be used to determine whether to move the lead 1914. If it is determined to move the lead, the method may use the comparison to determine where to the move the lead 1915. Similarly, rather than or in addition to moving the lead (including advancing or rotating the lead), the comparison may be used to adjust the stimulation settings. Various inputs, such as clinical outcomes, operating room-specific variables, and the like may be used to refine the ER target 1916. Machine learning algorithms may be implemented for the process to refine the ER target.

FIG. 20 illustrates, by way of example and not limitation, a method for determining an ER target and comparing sensed ERs to the determined ER target. At 2017, a system may use specifics and analytics to generate an ER target (which may include target features) 2018 by determining and analyzing data such as the patient's anatomical data or other electrophysiological or behavior data 2019, and the knowledge base of the center and surgeon 2020. At 2021, the method may sense and record ERs to provide ERs or feature(s) of ERs 2022. At 2023, the ERs are compared to the target to determine whether the difference is within a user-determined bounds. Some embodiments may also consider whether a maximum number of steps have been taken. If the difference is not with in the user-determined bounds, the lead may be moved to reduce the difference between the recorded ER (features) and the evoked response target (features) 2024, and the process may return to 2022 to continue to check the ERs. Some embodiments may use the sensed EP response and features into the target/template specifics and analytics 2017 to update the ER target.

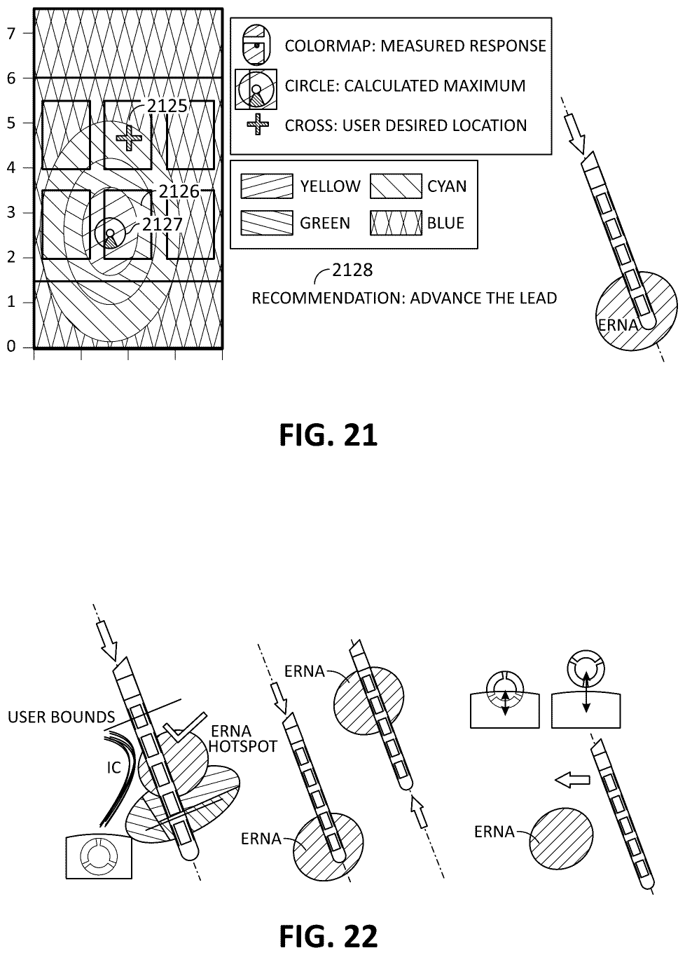

FIG. 21 illustrates, by way of example and not limitation, a display on a user interface for identifying a user defined location for the ER (ER target) 2125, a sensed ER 2126 including a calculated maximum for the sensed ER 2127, and a recommendation 2128 for the user to cause the sensed ER to more closely correspond to the ER target. The responses target may be presented on a representation of lead electrodes. In the illustrated embodiment, the recommendation 2124 is to advance the lead to cause the maximum of the ER to move closer to the target.

FIG. 22 illustrates, by way of example, elements that may be presented on a display in a user interface that provide a simple and clear confirmation of lead position. The target may be specific to anatomy, surgery center, and surgeon. The target may be updated, such as with analytics or machine learning, and may combine patient outcomes with patient biopotential recordings. The desired hotspot for ERs may be represented using the ERNA ovals, where ERNA is maximum. The user may provide some bounds along the lead to identify what is an acceptable response The bounds may be based on prior knowledge, such as but not limited to patient data from a group of patients in a center. The illustrated interface may include an indicator for suggesting a move (e.g., arrows) based the relative locations of the measured to desired responses. The movement suggestions may include moving the lead up/down moving the lead longitudinally, translating the lead body, or rotating the lead. The response may be determined to be acceptable when the hotspot of the sensed ERs is in the same or nearly the same position (within bounds) as the ER target. The response may be determined to be acceptable when the distribution of the sensed ERs is determined to be similar to the distribution of the target. The comparison of the distribution may be based on various statistical measures of distribution, or may be based on a visual appearance to the user.

The ER distributions may include longitudinal distributions along the lead and directional distributions about the lead. For example, the ER target can be a desired direction distribution such as but not limited to one electrode segment in a segmented electrode results in ERNA 3× stronger than ERNA of another electrode segment in the segmented electrode. The system may allow the user to select the optimal target or may provide a default target and allow the user to edit the default target.

A surgery center may generate or update a sweet spot map by collecting data from a series of patients using an operating room system. Operating room recordings and clinical responses may be aggregated. A sweet spot map may be determined specifically for the center. The recordings and responses may be associated through imaging, and may be scored based on outcomes. The system may be configured to import sweet spot mapping data from other patients and centers. The sweet spot maps may be updated with subsequent patients.

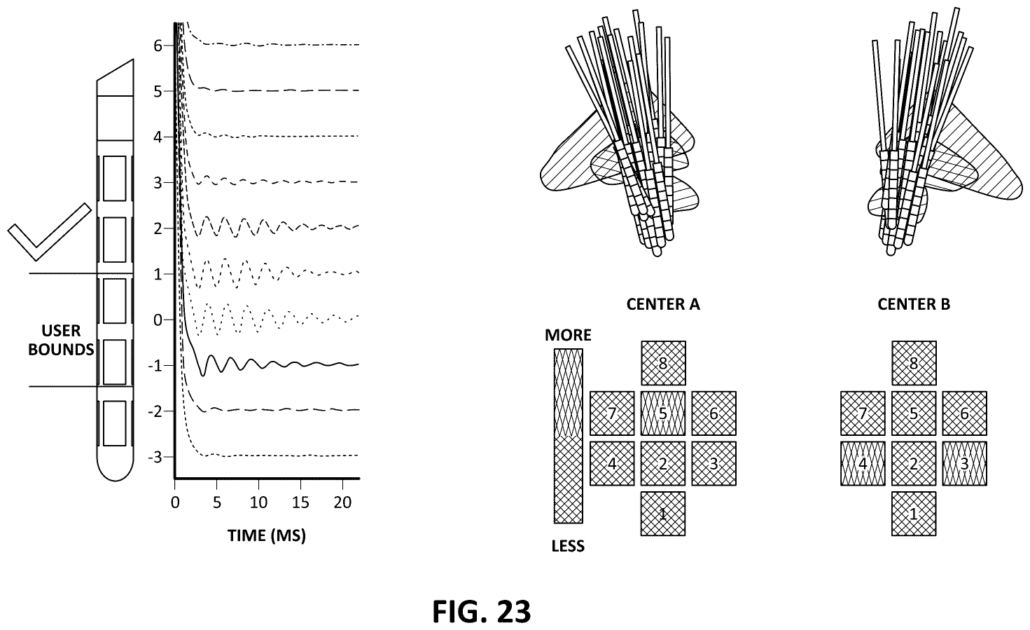

FIG. 23 illustrates that the ER target may be specific to the surgery center used to perform the DBS lead implant. Different surgery centers may have significant differences in approaches to implanting a DBS lead and imaging the area, even when stimulation is being delivered to the same anatomical target. For example, different surgery centers may use different imaging techniques and different frames for guiding the implantation of the lead. These differences can result in different sensed ERs. By way of example, each center may have their preferred trajectory angles for the DBS leads as illustrated in FIG. 23. As a result of this and other preferences such as DBS lead type, DBS target, DBS lead entry point, and the like, the desired ER target for Center A may correspond to electrode segment 5, and the desired ER target for Center B may correspond to electrode segments 3 and 4. For example, the system may be configured to track the most-used settings to generate user-specific ER targets. Some embodiments associate the ER targets with outcomes to further refine the targets. FIG. 23 also illustrate a plurality of sensed response waveforms along the lead. The user may provide user bounds along the lead to identify what is the bounds for an acceptable response. The user bounds may be informed by anatomy or other electrophysical or behavioral data.

A feature profile for a desired ER target may differ depending on the target for the stimulation. For example, even if the same electrodes on the lead are used, the feature profile for the desired ER target to (PSA+STN)-DBS may be skewed from the desired ER target to GPi-DBS. (PSA+STN)-DBS is DBS applied to target the posterior subthalamic area (PSA) and the subthalamic nucleus (STN). GPi-DBS is DBS applied to target the internal globus pallidus (GPi). Furthermore, a desired ER target may change over time for delivering DBS to the same anatomical target at the same center.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encrypted with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method, comprising:
delivering, using a controller, electrostimulation from an electrostimulator in accordance with a stimulation setting;
determining, using the controller and a sensing circuit configured to sense evoked responses (ERs), sensed ERs to the electrostimulation delivered in accordance with a sensing setting and the stimulation setting;
determining acceptance criteria using user input received via a user interface;
comparing, using the controller, the sensed ERs to the acceptance criteria to provide a comparison result; and
displaying an indicator of the comparison result on the user interface.

2. The method of claim 1, wherein the electrostimulator is configured to provide electrostimulation to a therapy target in a brain of the patient or other preferred location via a deep brain stimulator (DBS) lead, and the evoked responses include Evoked Resonant Neural Activity (ERNA).

3. The method of claim 1, further comprising receiving, using the user interface, user-provided acceptance bounds for the sensed ERs, wherein the acceptance criteria include the user-provided acceptance bounds.

4. The method of claim 1, further comprising receiving a user selection of a target ER template from stored ER templates, wherein the acceptance criteria include the target ER template, and the comparing includes comparing the sensed ERs to the target ER template to provide the comparison result.

5. The method of claim 4, wherein:
the stored ER templates are stored within storage of an operating room (OR) system configured to be used during a lead placement procedure, the OR system including at least the electrostimulator, the sensing circuit, the user interface; or
the stored ER templates are stored in a cloud-based storage in a cloud-computing system.

6. The method of claim 1, wherein the displaying the indicator of the comparison result includes displaying on the user interface representations of the acceptance criteria and the sensed ERs and/or derivatives thereof.

7. The method of claim 1, further including:
displaying on the user interface a representation of the lead; and
determining and displaying a suggested lead movement to cause the sensed ERs to compare more favorably to the acceptance criteria.

8. The method of claim 1, further including determining and displaying on the user interface a suggestion for initializing and/or changing the stimulation setting or the sensing setting to cause the sensed ERs to compare more favorably to the acceptance criteria.

9. The method of claim 1, further including providing ER templates available for user selection by modifying ER templates or creating new ER templates based on sensed ERs and patient outcome for one or more patients, wherein the acceptance criteria include a user-selected ER template, and wherein the providing ER templates includes implementing one or more machine learning algorithms to:
modify or create the ER templates by determining relationships among data where the data include the sensed ERs, the patient outcomes, and at least one lead implant procedure input; and
determine the sensed ERs that corresponds to a desirable patient outcome when electrostimulation is delivered to the neural target.

10. The method of claim 9, wherein the machine learning algorithm is implemented using at least one of a cloud-based application or a deployed system.

11. The method of claim 1, further comprising indexing the acceptance criteria by region, clinical institution, group, or participants information, wherein the acceptance criteria are based at least in part on an identification of an institution where the patient is implanted or treated with the electrostimulation, a group, or the participants information.

12. The method of claim 1, further comprising indexing the acceptance criteria by implanter information, wherein the acceptance criteria are based at least in part on an identification of an implanter that implants the lead.

13. The method of claim 1, further comprising indexing the acceptance criteria by symptom relief goals, wherein the acceptance criteria are based at least in part on the sensed indication of symptom relief of the patient, wherein the symptom relief goals include at least one of a type motor function or a type of cognitive function.

14. The method of claim 1, wherein the acceptance criteria include a target distribution of ER data, the determining sensed ERs includes determining a distribution of sensed ERs, the comparing includes comparing the distribution of sensed ERs to the distribution of ER data, and the displaying the indicator includes displaying the distribution of sensed ERs and the target distribution of ER data.

15. The method of claim 1, wherein the acquisition criteria include at least one target representative ER feature, the determining sensed ERs includes determining at least one measured ER feature from the sensed ERs, and the comparing includes comparing the at least one target representative ER feature to the at least one measured ER feature.

16. A non-transitory machine-readable medium including instructions, which when executed by a machine, cause the machine to perform a method comprising:
delivering, using a controller, electrostimulation from an electrostimulator in accordance with a stimulation setting;
determining, using the controller and a sensing circuit configured to sense evoked responses (ERs), sensed ERs to the electrostimulation delivered in accordance with a sensing setting and the stimulation setting;
determining acceptance criteria using user input received via a user interface;
comparing, using the controller, the sensed ERs to the acceptance criteria to provide a comparison result; and
displaying an indicator of the comparison result on the user interface.

17. A system, comprising:
an electrostimulator configured to provide electrostimulation to a neural target patient via electrodes on a lead;
a sensing circuit configured to sense, at a plurality of sensing locations, evoked responses (ERs) to the electrostimulation;
a user interface; and
a controller operably connected to the electrostimulator, the sensing circuit and the user interface, and configured to:
deliver the electrostimulation in accordance with a stimulation setting;
determine the sensed ERs to the electrostimulation delivered in accordance with a sensing setting and the stimulation setting;
determine acceptance criteria using user input received via the user interface;
compare the sensed ERs to the acceptance criteria to provide a comparison result; and
display on the user interface an indicator of the comparison result.

18. The system of claim 17, wherein the electrostimulator is configured to provide electrostimulation to a therapy target or other preferred lead placement location in a brain of the patient via a deep brain stimulator (DBS) lead, and the evoked responses include Evoked Resonant Neural Activity (ERNA).

19. The system of claim 17, wherein the controller is configured to receive, using the user interface, user-provided acceptance bounds for the sensed ERs, and the acceptance criteria include the user-provided acceptance bounds.

20. The system of claim 17, further comprising a storage system configured to store a plurality of ER templates, the ER templates each representing one or more of a single, patient-specific response or a population-based response to electrostimulation of the neural target, wherein the user input received via the user interface includes a user selection of a target ER template from the stored ER templates, the acceptance criteria include the target ER template, and the controller is configured to compare the sensed ERs to the target ER template to provide the comparison result.
